(19) United States
(12) Patent Application Publication
(10) Pub. No.: US 2025/0073162 A1
(43) Pub. Date: Mar. 6, 2025

(54) A STABLE INJECTABLE COMPOSITION OF TRIAMCINOLONE ACETONIDE

(71) Applicant: INDOCO REMEDIES LIMITED, Rabale, Navi Mumbai (IN)

(72) Inventors: Aditi PANANDIKAR, Rabale, Navi Mumbai (IN); Sundeep BAMBOLKAR, Rabale, Navi Mumbai (IN); Kavita INAMDAR, Rabale, Navi Mumbai (IN); Gopal WAWDE, Rabale, Navi Mumbai (IN); Chetan GUNDECHA, Rabale, Navi Mumbai (IN); Swati SAWANT, Rabale, Navi Mumbai (IN)

(21) Appl. No.: 18/846,541

(22) PCT Filed: Feb. 27, 2023
(86) PCT No.: PCT/IB2023/051789

§ 371 Date: Sep. 12, 2024

(30) Foreign Application Priority Data
Mar. 17, 2022 (IN) 202221014752

(51) Int. Cl. A61K 9/10; A61K 9/00; A61K 31/58; A61K 47/26; A61K 47/38

(52) U.S. Cl. CPC A61K 9/10; A61K 9/0019; A61K 31/58; A61K 47/26; A61K 47/38

(57) ABSTRACT

The present invention relates to a stable injectable composition of Triamcinolone acetonide and a method of preparing same, which is stable at controlled room temperature between 200 C-250 C and packaged in glass material. Specifically, the triamcinolone acetonide injection of the present invention shows improved viscosity within range of 10-30 cps and osmolality within range of 270 to 370 mOsm/kg.

### FIELD OF INVENTION

The present invention relates to a stable injectable composition comprising triamcinolone acetonide. More particularly, the present invention relates to a stable injectable suspension comprising triamcinolone acetonide and pharmaceutically acceptable excipients. The present invention also relates to a process for the preparation of stable injectable suspension comprising triamcinolone acetonide and its use for treatment of various allergic conditions and organ disorders such as anemia hemolytic anemia, Diamond-blackfan anemia, ulcerative colitis and many other disorders.

### BACKGROUND OF THE INVENTION

An adrenal cortex produces steroid hormones and these are categorized as corticosteroids which are further sub-categorized as glucocorticoids. Many feedback mechanisms are present in the human body to evoke the immune system and one of them is a glucocorticoid-based system that aids to reduce inflammation, therefore glucocorticoids are mostly preferred for treating inflammatory diseases. Triamcinolone acetonide is a glucocorticoid used to treat various disease conditions. It is a synthetic glucocorticoid with anti-inflammatory and immunomodulating properties.

Triamcinolone acetonide has better efficiency than hydrocortisone or cortisone drugs. This drug is useful to treat a number of medical conditions like allergies, sclerosis, lupus, ocular inflammation, central retinal vein occlusion etc. The USFDA has approved various dosage forms such as injection, spray, cream, lotion, syrup, paste, and ointment for treating various disease conditions in humans. The Kenalog 40 injection which is approved by USFDA, is administered via intramuscular or intra-articular route.

Chinese patent application number CN104971039 discloses triamcinolone acetonide acetate solution comprising of hyaluronic acid sodium, sulfobutyl ether, β cyclodextrin and other excipients. The other Chinese Patent application CN101618019 discloses the manufacturing method for triamcinolone acetonide acetate microparticles and tried to solve problems related to the in-process crystallization of APL

The application number US20050065137 focuses on triamcinolone suspension composition comprising of polyvinylpyrrolidone, tonicity adjusting agent, buffering agent and pH adjusting agent and water for injection for treatment of eye. This composition is devoid of both preservative as well as surfactant. The final product has a pH between 6-8 and viscosity of 50 cps.

The U.S. Pat. No. 8,846,094B2 discloses the use of triamcinolone injection composition for treating arthritis. The said composition comprises polymeric hyaluronate or polymeric hyaluronic acid and cyclodextrin.

The Triamcinolone acetonide containing pharmaceutical dosage forms are disclosed in various prior art documents, however very few emphasize on to improve the stability of dosage form by modification in the manufacturing process.

The major challenge in the manufacturing of the triamcinolone acetonide injectable suspension is to achieve desired viscosity and to retain the viscosity on stability, storage and during the shelf life of the suspension. Being a sterile dosage form, the challenge was to select suitable sterilization technique that will achieve the sterility of viscosity modifying agent and also maintain the viscosity of the injectable suspension.

Filtration through a 0.22 microns filter is a common approach to sterilise solution. Viscosity modifying agents like carboxymethylcellulose sodium being soluble in aqueous medium, filtration approach was tried for different dilutions. However, due to the chemistry of the carboxymethylcellulose sodium, a crosslinked system of polymer was formed which was difficult to filter.

In dry heat sterilization using hot air oven, exposure to higher time and temperature resulted in drop in the initial viscosity. Hence, this approach was not considered.

Gaseous sterilization using ethylene oxide resulted in the increased viscosity than the desired value but resulted in the drop in the viscosity on stability. Additionally, this technique of sterilization takes longer timer, and would increase an additional testing of quantification of residual gases.

Gamma sterilization approach was also tried but the finished formulations resulted in lower viscosity than desired. Also, this sterilization technique resulted in changes in the physicochemical properties of the sodium CMC.

Moist heat sterilization using autoclave was tried to sterilize the viscosity modifying agents like carboxymethylcellulose sodium in solution form, which resulted in achieving the desired viscosity but due to exposure to high temperature and pressure during autoclave, viscosity of the final suspension product was found to be unstable on storage. However, attempts were made to optimize this process being the most common approach of sterilization.

Surprisingly, the inventors of the present invention have found a novel process for preparing triamcinolone acetonide injection which not only improves viscosity but also stability of finished dosage form. The viscosity of triamcinolone acetonide injectable suspension composition remains within the desirable range of 10-30 cps.

### OBJECT OF THE INVENTION

The object of the present invention is to provide a stable injectable composition comprising triamcinolone acetonide and pharmaceutically acceptable excipients.

Another object of the present invention is to provide a stable injectable composition comprising triamcinolone acetonide, preservative agent and pharmaceutically acceptable excipients.

Another object of the present invention is to provide a stable injectable composition comprising triamcinolone acetonide, preservative agent, viscosity modifying agent, pH adjusting agent and pharmaceutically acceptable excipients.

Yet another object of the invention is to provide a stable injectable composition comprising Triamcinolone acetonide, preservative agent, viscosity modifying agent, pH adjusting agent and has viscosity in the range of 10-30 cps.

Further object of the present invention is to provide a process for preparation of a stable injectable dosage form comprising triamcinolone acetonide.

Still, a further object of the present invention is to provide a process for the preparation of a stable suspension of triamcinolone acetonide injectable dosage form with better viscosity that will remain in the acceptable range during stability, storage or entire shelf life.

### SUMMARY OF THE INVENTION

The present invention relates to a stable injectable composition comprising triamcinolone acetonide and pharmaceutically acceptable excipients.

The present invention further relates to a process for the preparation of a stable injectable suspension comprising of following steps:
a) Preparing polymer phase comprising of tonicity adjusting agent, viscosity modifying agent and water.
b) Adding preservative to polymer phase of step a) and adjusting pH with pH adjusting agent, followed by moist heat sterilization.
c) Preparing a solution comprising of wetting agent and water, followed by sterilization by filtration.
d) Preparing API phase by mixing solution of step (c) and triamcinolone acetonide followed by homogenization to reduce particle size in the range average of 7 μm to 15 μm.
e) Mixing both polymer phase of step (b) and API phase of step (d), followed by final pH adjustment and volume make up to prepare a homogeneous suspension of triamcinolone acetonide.

### DETAILED DESCRIPTION OF THE INVENTION

In one aspect, the present invention provides a stable injectable composition comprising triamcinolone acetonide and pharmaceutically acceptable excipients.

According to one embodiment, the present invention provides a stable injectable composition comprising triamcinolone acetonide and preservative agent.

According to one embodiment, the present invention provides a stable injectable composition comprising triamcinolone acetonide, preservative agent, viscosity modifying agent, wetting agent, tonicity agent and pharmaceutically acceptable excipients.

According to one embodiment, the present invention provides a stable injectable composition comprising triamcinolone acetonide, benzyl alcohol, carboxymethylcellulose sodium, polysorbate 80, sodium chloride and pharmaceutically acceptable excipients.

In one embodiment, the injectable composition comprises triamcinolone acetonide, preservative agent, viscosity modifying agent, wetting agent and pH adjusting agent, wherein the composition is sterile.

According to one embodiment, the present invention provides a stable injectable composition comprising triamcinolone acetonide 1% to 8%, tonicity adjusting agent 0.01% to 1%, viscosity modifying agent 0.25% to 2.5%, wetting agent 0.02% to 2%, preservative 0.001% to 2%, pH adjusting agent and water for injection.

According to one embodiment, the present invention provides a stable injectable composition comprising triamcinolone acetonide 1% to 8%, Sodium chloride 0.01% to 1%, carboxymethylcellulose sodium 0.25% to 2.5%, polysorbate 80 0.02% to 2%, benzyl alcohol 0.001 to 2%, sodium hydroxide, hydrochloric acid and water for injection.

According to one embodiment, the present invention relates to a pH adjustment of polymer phase to prepare pharmaceutical injectable composition comprising Triamcinolone acetonide and other pharmaceutical excipients.

According to one embodiment, the present invention provides a stable injectable composition comprising 1 to 8% w/v triamcinolone acetonide, 0.001 to 2% w/v preservative, 0.01 to 1% w/v tonicity agent, 0.25 to 2.5% w/v viscosity modifying agent, 0.02 to 2% w/v wetting agent, and pH adjusting agent, wherein said composition has pH in the range of 5 to 8, viscosity in the range of 10 to 30 cps, stable over time at controlled room temperature between 20° C. to 25° C. and compatible to the glass packaging material.

According to one embodiment, the present invention provides a stable injectable composition comprising 1 to 8% w/v triamcinolone acetonide, 0.001 to 2% w/v benzyl alcohol, 0.01 to 1% w/v sodium chloride, 0.25 to 2.5% w/v carboxymethylcellulose sodium, 0.02 to 2% w/v polysorbate 80, and sodium hydroxide and/or hydrochloric acid; wherein the said composition has pH in the range of 5 to 8 and stable over time at controlled room temperature between 20° C. to 25° C. and compatible to the glass packaging material.

According to one embodiment, the present invention provides a stable injectable composition comprising 4% w/v triamcinolone acetonide, 0.99% w/v preservative, 0.66% w/v tonicity agent, 0.63% w/v viscosity modifying agent, 0.04% w/v wetting agent and pH adjusting agent.

According to one embodiment, the present invention provides a stable injectable composition comprising 4% w/v triamcinolone acetonide, 0.99% w/v benzyl alcohol, 0.66% w/v sodium chloride, 0.63% w/v carboxymethylcellulose sodium, 0.04% w/v polysorbate 80, and sodium hydroxide and/or hydrochloric acid.

According to one embodiment, the present invention provides a stable injectable composition comprising 4% w/v triamcinolone acetonide, 0.99% w/v preservative, 0.66% w/v tonicity agent, 0.63% w/v viscosity modifying agent, and 0.04% w/v wetting agent; wherein the composition has final viscosity between 10-30 cps and pH between 5 to 8.

According to one aspect, the present invention provides a preservative free stable injectable composition comprising triamcinolone acetonide, and pharmaceutically acceptable excipients.

According to one embodiment, the present invention provides a preservative free stable injectable composition comprising triamcinolone acetonide, tonicity adjusting agent, viscosity modifying agent, wetting agent, pH adjusting agent and water for injection.

According to one embodiment, the present invention provides a preservative free stable injectable composition comprising triamcinolone acetonide, sodium chloride, carboxymethylcellulose sodium, polysorbate 80, sodium hydroxide, hydrochloric acid and water for injection.

According to another aspect, the present invention provides a method of prevention or treatment of various diseases by administering to patient a stable injectable composition triamcinolone acetonide.

According to one embodiment, the present invention provides a method of treating allergic conditions such as incapacitating allergic conditions intractable to adequate trials of conventional treatment in asthma, atopic dermatitis, contact dermatitis, drug hypersensitivity reactions, perennial or seasonal allergic rhinitis, serum sickness, and transfusion reactions, by administering a composition comprising of triamcinolone acetonide and other pharmaceutical acceptable excipients via intramuscular route.

According to one embodiment, the present invention provides a method of treating dermatologic diseases such as Bullous dermatitis herpetiformis, exfoliative erythroderma, mycosis fungoides, pemphigus, severe and erythema multiforme (Stevens-Johnson syndrome), by administering a composition comprising of triamcinolone acetonide and other pharmaceutical acceptable excipients via intramuscular route.

According to one embodiment, the present invention provides a method of treating endocrine disorders such as primary or secondary adrenocortical insufficiency (hydrocortisone or cortisone is the drug of choice; synthetic analogs may be used in conjunction with mineralocorticoids where applicable; in infancy, mineralocorticoid supplementation is of particular importance), congenital adrenal hyperplasia, hypercalcemia associated with cancer, nonsuppurative thyroiditis, by administering a composition comprising of triamcinolone acetonide and other pharmaceutical acceptable excipients via intramuscular route.

According to one embodiment, the present invention provides a method of treating gastrointestinal diseases such to tide the patient over a critical period of the disease in regional enteritis and ulcerative colitis, by administering a composition comprising of triamcinolone acetonide and other pharmaceutical acceptable excipients via intramuscular route.

In one embodiment, the present invention provides a method of treating hematologic disorders such as acquired (autoimmune) hemolytic anemia, diamond-blackfan anemia, pure red cell aplasia, selected cases of secondary thrombocytopenia, by administering a composition comprising of triamcinolone acetonide and other pharmaceutical acceptable excipients via intramuscular route.

According to one embodiment, the present invention provides a method of treating miscellaneous conditions such as trichinosis with neurologic or myocardial involvement, tuberculous meningitis with subarachnoid block or impending block when used with appropriate antituberculosis chemotherapy, by administering a composition comprising of triamcinolone acetonide and other pharmaceutical acceptable excipients via intramuscular route.

According to one embodiment, the present invention provides a method of managing neoplastic diseases like the palliative management of leukemias and lymphomas, by administering a composition comprising of triamcinolone acetonide and other pharmaceutical acceptable excipients via intramuscular route.

According to one embodiment, the present invention provides a method of treating nervous system such as acute exacerbations of multiple sclerosis; cerebral edema associated with primary or metastatic brain tumor or craniotomy, by administering a composition comprising of triamcinolone acetonide and other pharmaceutical acceptable excipients via intramuscular route.

According to one embodiment, the present invention provides a method of treating ophthalmic diseases such as sympathetic ophthalmia, temporal arteritis, uveitis, and ocular inflammatory conditions unresponsive to topical corticosteroids, by administering a composition comprising of triamcinolone acetonide and other pharmaceutical acceptable excipients via intramuscular route.

According to one embodiment, the present invention provides a method of treating renal diseases such as induce diuresis or remission of proteinuria in idiopathic nephrotic syndrome or that due to lupus erythematosus, by administering a composition comprising of triamcinolone acetonide and other pharmaceutical acceptable excipients via intramuscular route.

According to one embodiment, the present invention provides a method of treating respiratory diseases such as berylliosis, fulminating or disseminated pulmonary tuberculosis when used concurrently with appropriate antituberculosis chemotherapy, idiopathic eosinophilic pneumonias and symptomatic sarcoidosis, by administering a composition comprising of triamcinolone acetonide and other pharmaceutical acceptable excipients via intramuscular route.

According to one embodiment, the present invention provides a method of treating rheumatic disorders such as adjunctive therapy for short-term administration (to tide the patient over an acute episode or exacerbation) in acute gouty arthritis; acute rheumatic carditis; ankylosing spondylitis; psoriatic arthritis; rheumatoid arthritis, including juvenile rheumatoid arthritis (selected cases may require low-dose maintenance therapy), dermatomyositis, polymyositis, and systemic lupus erythematosus, by administering a composition comprising of triamcinolone acetonide and other pharmaceutical acceptable excipients via intramuscular route. According to one embodiment, the present invention provides a method of treating acute gouty arthritis, acute and subacute bursitis, acute nonspecific tenosynovitis, epicondylitis, rheumatoid arthritis, synovitis of osteoarthritis, by administering a composition comprising of triamcinolone acetonide and other pharmaceutical acceptable excipients via intra-articular route.

According to another aspect, the present invention provides a stable injectable composition comprising triamcinolone acetonide and pharmaceutically acceptable excipients packaged in pharmaceutically acceptable containers. According to one embodiment, the present invention provides a stable injectable composition comprising triamcinolone acetonide and pharmaceutically acceptable excipients packaged in glass vial or glass ampoule.

According to one embodiment, the present invention provides a stable injectable composition comprising triamcinolone acetonide and pharmaceutically acceptable excipients packaged in clear siliconized USP type I glass vials.

According to one embodiment, the present invention provides a stable injectable composition comprising triamcinolone acetonide and pharmaceutically acceptable excipients packaged in clear sulphur treated USP type I glass vials.

According to one embodiment, the present invention provides a stable injectable composition comprising triamcinolone acetonide and pharmaceutically acceptable excipients packaged in amber color USP type I glass vials.

The pharmaceutically acceptable excipients contained in the stable injectable composition of the present invention include preservative agents, tonicity adjusting agents, viscosity modifying agents, wetting agents, pH adjusting agents and other conventional agents that may be typically used in formulating an injectable composition.

Tonicity adjusting agents that can be used in the stable injectable composition of the present invention include, but are not limited to potassium chloride, sodium chloride, glycerin dextrose, mannitol, sorbitol, lactose, glycerol and combinations thereof.

Viscosity modifying agents that can be used in the stable injectable composition of the present invention include, but are not limited to povidone, carboxymethylcellulose sodium, polyvinylpyrrolidone compounds, polyethylene glycols, acacia, gelatin, methyl cellulose and combinations thereof.

Wetting agents that can be used in the stable injectable composition of the present invention include, but are not limited to lecithin, polysorbate 20, sorbitan monolaurate, polysorbate 80 and combinations thereof.

Preservative agents that can be used in the stable injectable composition of the present invention include, but are not limited to methylparaben, propylparaben, benenzalkonium chloride, thiomersal, benzyl alcohol and combinations thereof.

pH adjusting agent that can be used in the stable injectable composition of the present invention include, but are not limited to acetate, citrate, tartrate, phosphate, sulphuric acid, sodium hydroxide, hydrochloric acid, arginine, phosphate glycine and combinations thereof.

The manufacturing process is one of the important factor and can affect the stability of finished pharmaceutical dosage form. Viscosity plays vital role to maintain stability of injection during shelf life.

The main factors affecting stability of pharmaceutical dosage form are qualitative and quantitative selection of ingredients, manufacturing process, packaging system and storage conditions.

Stability of any injectable dosage form is dependent on the satisfactory physicochemical parameters obtained at a time of manufacturing and maintaining those physicochemical parameters after sterilization. Hence selection of appropriate sterilization technique is important. In current manufacturing process two sterilization steps are involved:
1) Polymer phase by steam sterilization; and
2) Sterilization of wetting agent by filtration method to which sterile API is added to make API phase.

Polymer phase sterilization by autoclaving is crucial as it may affect the viscosity parameter of final product. Viscosity of carboxymethylcellulose sodium is inversely proportional to temperature and hence increase in temperature results in decrease in viscosity of solution; in addition to this, pH of solution plays important role in maintaining viscosity and hence heat sterilization of polymer phase needs to be handled cautiously to prepare injectable suspension with desired viscosity. Before sterilization maintaining pH values of carboxymethylcellulose sodium solution between 6.5 to 11, not only achieves desired viscosity of polymer phase but also good stability in final product. Prior to sterilization if pH is maintained at 6 viscosity of the solution rapidly decreases with formation of carboxy methyl celluose acid. However, if pH is maintained above 9, viscosity decreases slowly. If pH value is maintained more than 11.5, a sharp decline in viscosity is seen which is due to increase in bonding of unsubstituted hydroxyl group and alkaline molecules which aids in the dispersion of cellulose. Accordingly inventor of the present invention, has invented the middle course to achieve steam sterilization of carboxymethylcellulose sodium solution without compromising viscosity with the aid of tonicity agent and pH adjusting agent.

Another aspect the present invention relates to a process for the preparation of a stable injectable composition comprising triamcinolone acetonide and pharmaceutically acceptable excipients.

In one embodiment, the present invention provides a process for the preparation of a stable injectable composition comprising:
a) Preparing polymer phase comprising of tonicity adjusting agent, viscosity modifying agent and water.
b) Adding preservative to polymer phase of step a) and adjusting pH with pH adjusting agent, followed by moist heat sterilization.
c) Preparing a solution comprising of wetting agent and water, followed by sterilization by filtration.
d) Preparing API phase by mixing solution of step (c) and triamcinolone acetonide followed by homogenization to reduce average particle size in the range of 7 μm to 15 μm.
e) Mixing both polymer phase of step (b) and API phase of step (d), followed by final pH adjustment and volume make up to prepare a homogenous suspension of triamcinolone acetonide.

In one embodiment the average particle size of wet milling phase or homogenizer phase is reduced to 5 μm to 20 μm, preferably 6 μm to 18 μm, more preferably around 7 μm to 15 μm.

Some of the particle size reduction equipment's commonly used for size reduction are microfluidisers, high pressure homogenizers, jet milling, ball milling, probe sonicators.

In one embodiment, the present invention provides a process for manufacturing the stable injectable composition comprising:
a) Preparing polymer phase comprising of tonicity adjusting agent, viscosity modifying agent and water.
b) Adding preservative to polymer phase of step a) and adjusting pH with pH adjusting agent, followed by moist heat sterilization.
c) Preparing a solution comprising of wetting agent and water, followed by sterilization by filtration.
d) Preparing API phase by mixing solution of step (c) and triamcinolone acetonide followed by homogenization to reduce average particle size in the range of 7 μm to 15 μm.
e) Mixing both polymer phase of step (b) and API phase of step (d), followed by final pH adjustment and volume make up to prepare a homogeneous suspension of triamcinolone acetonide.
f) Aseptically filling a triamcinolone acetonide suspension of step e) in suitable sterile packaging means.

In one embodiment, the present invention provides a process for manufacturing the stable injectable composition comprising:
a) Preparing polymer phase comprising of tonicity adjusting agent, viscosity modifying agent and water.
b) Adding preservative to polymer phase of step a) and adjusting pH with pH adjusting agent, followed by moist heat sterilization at 121° C. and at 15 psi pressure for 15 minutes.
c) Preparing a solution comprising of wetting agent and water, followed by sterilization by filtration.
d) Preparing API phase by mixing solution of step (c) and triamcinolone acetonide followed by homogenization to reduce particle size in the range of 7 μm to 15 μm.
e) Mixing both polymer phase of step (b) and API phase of step (d), followed by final pH adjustment and volume make up to prepare a homogeneous suspension of triamcinolone acetonide.

In one embodiment, the present invention provides a process for manufacturing the stable injectable composition comprising:
a. Preparing polymer phase comprising of sodium chloride, carboxymethylcellulose sodium and water.
b. Adding preservative to polymer phase of step a) and adjusting pH with sodium hydroxide or hydrochloric acid, followed by moist heat sterilization at 121° C. and at 15 psi pressure for 15 minutes.
c. Preparing a solution comprising of polysorbate 80 and water, followed by sterilization by filtration.
d. Preparing API phase by mixing solution of step (c) and triamcinolone acetonide followed by homogenization to reduce average particle size in the range of 7 μm to 15 μm.
e. Mixing both polymer phase of step (b) and API phase of step (d), followed by final pH adjustment and volume make up to prepare a homogeneous suspension of triamcinolone acetonide.

The particle size of API phase is given in Table No. 1

| | Before homogenization | After homogenization |
|---|---|---|
| Parameter | (μm) | (μm) |
| D10 | 2.158 | 1.84 |
| D50 | 7.157 | 6.31 |
| D90 | 20.21 | 15.90 |

Different kinds of processes are used to manufacture stable injectable suspension and following experimental data represents the roadmap to achieve product of superior quality.

### Experiment No. 1 (Batch No. TAS/R/40/104): Without Using Sodium Hydroxide in Polymer Phase and Only Complete Batch Homogenization

The injectable suspension is prepared by following process:
1. Polymer phase—Under constant stirring carboxymethylcellulose sodium was dissolved in water for injection. Benzyl alcohol was added to carboxymethylcellulose sodium solution. Sterilization of polymer phase by moist heat sterilization.
2. API phase—Sodium chloride and Polysorbate 80 were dissolved in water and sterilized through filter. Added triamcinolone acetonide to sterilized solution.
3. Final injectable suspension—Aseptically mixed polymer phase with API phase. pH of mixed suspension was adjusted with Hydrochloric acid or sodium hydroxide solution or combination of both agents. Final suspension volume was prepared, homogenized and packed in suitable packing material.

TABLE NO 2
Stability data of Triamcinolone Acetonide Injectable Suspension (Batch No: TAS/R/40/104)
at 40° C./75% RH (Accelerated condition) and 25° C./60% RH (Long term condition)

| | | Batch No. | | | | | | |
|---|---|---|---|---|---|---|---|---|
| | | TAS/R/40/104 | | | | | | |
| | Acceptance | | 40° C./75% RH | | | | 25° C./60% RH) | |
| Condition | limit | Initial | 1 M | 2 M | 3 M | 6 M | 3 M | 6 M |
| Appearance | White to off- | White to off- | White to off- | White to off- | White to off- | White to off- | White to off- | White to off- |
| | white aqueous | white aqueous | white aqueous | white aqueous | white aqueous | white aqueous | white aqueous | white aqueous |
| | suspension | suspension | suspension | suspension | suspension | suspension | suspension | suspension |
| pH | Between | 6.00 | 5.90 | 5.87 | 5.74 | 5.60 | 5.97 | 5.91 |
| | 5 and 7.5 | | | | | | | |
| Osmolality | 270 to 370 | 297 | 300 | 284 | 292 | 287 | 290 | 288 |
| Viscosity | 10 to 30 cps | 11.0 | 7.41 | 6.06 | 5.49 | 4.17 | 9.03 | 7.23 |
| (RPM 20, | | | | | | | | |
| Spindle S00) | | | | | | | | |

### Conclusion

Based on the 6 months stability data at accelerated and long term storage condition, it is observed that, Triamcinolone Acetonide Injectable suspension showed no change in the appearance and osmolality.

Further pH showed decreasing tendency during the accelerated condition however it was stable at long term conditions.

Viscosity of this formulation obtained was lower than the desired viscosity of 15 to 30 cps at initial and showed tendency to drop further on accelerated and long-term storage conditions.

### Experiment No. 2 (Batch No. TAS/R/40/105): Without Using Sodium Hydroxide in Polymer Phase and Only API Phase Homogenization

The injectable suspension is prepared by following process:
1. Polymer phase—Under constant stirring carboxymethylcellulose sodium was dissolved in water for injection. Benzyl alcohol was added to carboxymethylcellulose sodium solution. Sterilization of polymer phase by heat sterilization.
2. API phase—Sodium chloride and Polysorbate 80 were dissolved in water and sterilized through filter. Added triamcinolone acetonide to sterilized solution. Then resulted solution was homogenized using homogenizer.
3. Final injectable suspension—Aseptically mixed polymer phase with API phase. pH of suspension was adjusted with Hydrochloric acid or sodium hydroxide solution or combination of both agents. Final suspension volume was prepared and packed in suitable packing material.

TABLE NO 3
Stability data of Triamcinolone Acetonide Injectable Suspension (Batch No: TAS/R/40/105) at 25° C./60% RH

| | | Batch No. | | | | | | |
|---|---|---|---|---|---|---|---|---|
| | | TAS/R/40/105 | | | | | | |
| | Acceptance | | 40° C./75% RH | | | | 25° C./60% RH) | |
| Condition | limit | Initial | 1 M | 2 M | 3 M | 6 M | 3 M | 6 M |
| Appearance | White to off- | White to off- | White to off- | White to off- | White to off- | White to off- | White to off- | White to off- |
| | white aqueous | white aqueous | white aqueous | white aqueous | white aqueous | white aqueous | white aqueous | white aqueous |
| | suspension | suspension | suspension | suspension | suspension | suspension | suspension | suspension |
| pH | Between | 5.95 | 5.86 | 5.85 | 5.81 | 5.55 | 6.00 | 5.93 |
| | 5 and 7.5 | | | | | | | |
| Osmolality | 270 to 370 | 333 | 331 | 323 | 326 | 315 | 323 | 310 |
| Viscosity | 10 to 30 cps | 21.0 | 12.0 | 7.44 | 6.24 | 4.32 | 14.9 | 12.0 |
| (RPM 20, | | | | | | | | |
| Spindle S00) | | | | | | | | |

NA: Not available

### Conclusion

Based on the 6 months stability data at long term storage condition, it is observed that, Triamcinolone Acetonide Injectable suspension showed no change in the appearance of the suspension.

Osmolaity was found to be slightly decreasing on storage.

Further pH showed decreasing tendency during the accelerated condition however it was stable at long term conditions

Viscosity of this formulation was in the desired range of 15 to 30 cps. However, rapid drop in the viscosity was seen in 1 month at accelerated storage condition.

### Experiment No—3 (Batch No. TAS/R/40/040 & TAS/R/40/043) Using Sodium Hydroxide in Polymer Phase

1. Polymer phase—Under constant stirring sodium chloride and carboxymethylcellulose sodium were dissolved in water for injection. Benzyl alcohol was added to carboxymethylcellulose sodium solution. Then pH of solution is adjusted using sodium hydroxide or hydrochloric acid solution alone or in combination. Sterilization of polymer phase is done by heat sterilization.
2. API phase—Polysorbate 80 was dissolved in water and sterilized by filtration using 0.22 μm filter. Added triamcinolone acetonide to sterilized solution. Then resulted suspension was homogenized using homogenizer.
3. Final injectable suspension—Aseptically mixed polymer phase with API phase. pH of suspension was adjusted with Hydrochloric acid or sodium hydroxide solution or combination of both agents. Final suspension volume was prepared and packed in suitable packing material.

### Stability Data:

Two batches of Triamcinolone Acetonide Injectable suspension were manufactured. One with batch size: 1 L (Batch no: TAS/R/40/040) and second reproducible batch with batch size: 4 L (TAS/R/40/043). The batches were subjected to accelerated and long term stability conditions at 40° C./75% RH and 25° C./60% RH to monitor the physical parameters. The stability data is tabulated in table 4 & 5.

TABLE NO 4
Stability data of Triamcinolone Acetonide Injectable Suspension (Batch
No: TAS/R/40/040) at 40° C./75% RH and 25° C./60% RH.

| | | Batch No. | | | | |
|---|---|---|---|---|---|---|
| | | TAS/R/40/040 | | | | |
| | Acceptance | | 40° C./75% RH | | | 25° C./60% RH |
| Condition | limit | Initial | 1 Month | 2 Month | 3 Month | 3 Month |
| Appearance | White to off- | White to off- | White to off- | White to off- | White to off- | White to off- |
| | white aqueous | white aqueous | white aqueous | white aqueous | white aqueous | white aqueous |
| | suspension | suspension | suspension | suspension | suspension | suspension |
| pH | Between | 6.05 | 5.75 | 5.59 | 5.45 | 5.62 |
| | 5 and 7.5 | | | | | |
| Osmolality | 270 to 370 | 305 | 326 | 328 | 322 | 320 |
| (Miliosmol/kg) | | | | | | |
| Viscosity (RPM | 10 to 30 cps | 25.6 | 16.2 | 12.4 | 10.3 | 21.9 |
| 20, Spindle S00) | | | | | | |

TABLE NO 5
Stability data of Triamcinolone Acetonide Injectable Suspension (Batch
No: TAS/R/40/043) at 40° C./75% RH and 25° C./60% RH.

| | | Batch No. | | | | |
|---|---|---|---|---|---|---|
| | | TAS/R/40/043 | | | | |
| | | Batch size | | | | |
| | | 4.0 L | | | | |
| | Acceptance | | 40° C./75% RH | | | 25° C./60% RH |
| Condition | limit | Initial | 1 Month | 2 Month | 3 Month | 3 Month |
| Appearance | White to off- | White to off- | White to off- | White to off- | White to off- | White to off- |
| | white aqueous | white aqueous | white aqueous | white aqueous | white aqueous | white aqueous |
| | suspension | suspension | suspension | suspension | suspension | suspension |
| pH | Between | 6.0 | 6.0 | 5.8 | 5.8 | 5.88 |
| | 5 and 7.5 | | | | | |
| Osmolality | 270 to 370 | 334 | 328 | 321 | 322 | 325 |
| (Miliosmol/kg) | | | | | | |
| Viscosity (RPM | 10 to 30 cps | 28.3 | 18.5 | 14.9 | 12.9 | 22 |
| 20, Spindle S00) | | | | | | |

### Conclusion

Both the batches, TAS/R/40/040 & TAS/R/40/043 exhibited similar stability behaviour at accelerated (40° C./75% RH) and long term (25° C./60% RH) storage conditions. pH of the suspension showed drop from 6.05 to 5.65 in batch no. TAS/R/40/040 whereas slight drop was observed from 6.00 to 5.88 in batch no. TAS/R/40/043. Osmolality was found to be in range for both the batches.

For batch No. TAS/R/40/040 viscosity drop from 25.6 cps to 10.3 cps at accelerated condition (40° C./75% RH) while it was marginal drop from 25.6 cps to 21.9 cps at long term condition (25° C./60% RH) was observed. In case of batch no. TAS/R/40/043, viscosity decreased from 28.3 cps to 12.9 cps at accelerated condition (40° C./75% RH) and 28.3 cps to 22.0 cps at long term condition (25° C./60% RH). Since, all the parameters were found to be satisfactory and viscosity and pH drop was within the criteria at long term storage conditions, the strategy was finalized. Hence manufacturing process of experiment no 3 (Batch no. TAS/R/40/040 & TAS/R/40/043) is worthy for preparing Triamcinolone Acetonide injection suspension.

Although the invention has been described with reference to specific embodiments, this description is not meant to be construed in a limiting sense. Various modifications of the disclosed embodiments, as well as alternate embodiments of the invention, will become apparent to person skilled in the art upon reference to the description. It is therefore contemplated that such modification can be made without departing from the spirit or scope of the present invention as defined.

The following examples are given by way of illustration of the present disclosure and should not be construed to limit the scope of present disclosure. It is to be understood that both the foregoing general description and the following detailed description are exemplary and explanatory only and are intended to provide further explanation of the subject matter.

### Example 1

| Sr. | | | |
|---|---|---|---|
| No. | Ingredients | Mg/ml | % w/w |
| 1 | Triamcinolone acetonide | 40 | 4 |
| 2 | Sodium chloride | 6.6 | 0.66 |
| 3 | Carboxymethylcellulose sodium | 6.3 | 0.63 |
| 4 | Polysorbate 80 | 0.4 | 0.04 |
| 5 | Benzyl alcohol | 9.9 | 0.99 |
| 6 | Sodium Hydroxide | q.s | q.s |
| 7 | Hydrochloric acid | q.s | q.s |
| 8 | Water for injection | q.s to 1 ml | q.s |

### Manufacturing Process—

1) In S.S tank, under stirring sodium chloride and carboxymethylcellulose sodium were dissolved in water under stirring.
2) Benzyl alcohol was added in step 1 solution under stirring. Then pH was adjusted with sodium hydroxide.
3) Polymer solution from step 2 was autoclaved using heat sterilization.
4) In a separate S.S. tank, Polysorbate 80 was dissolved in water and sterilized through filter, to this triamcinolone acetonide was added.
5) The API phase of step 4 is homogenized using homogenizer.
6) The homogenized solution of step 4 is aseptically mixed with polymer phase of step 3.
7) pH is adjusted with hydrochloric acid or sodium hydroxide, if required.
8) Volume was made up with water and further filled and packed in suitable container.

### Example No. 2

| Sr. No. | Ingredients | Mg/ml | % w/w |
|---|---|---|---|
| 1 | Triamcinolone acetonide | 40 | 4 |
| 2 | Sodium chloride | 6.6 | 0.66 |
| 3 | Carboxymethylcellulose sodium | 7.5 | 0.75 |
| 4 | Polysorbate 80 | 0.4 | 0.04 |
| 5 | Benzyl alcohol | 9.9 | 0.99 |
| 6 | Sodium Hydroxide | q.s | q.s |
| 7 | Hydrochloric acid | q.s | q.s |
| 8 | Water for injection | q.s to 1 ml | q.s |

### Manufacturing Process—

1) In S.S tank, under stirring sodium chloride and carboxymethylcellulose sodium were dissolved in water.
2) Benzyl alcohol was added in step 1 solution. Then pH was adjusted with sodium hydroxide.
3) Polymer solution from step 2 was autoclaved using heat sterilization.
4) In a separate S.S. tank, Polysorbate 80 was dissolved in water and sterilized through filter, to this triamcinolone acetonide was added.
5) The API phase of step 4 is homogenized using homogenizer.
6) The homogenized solution of step 4 is aseptically mixed with polymer phase of step 3.
7) pH is adjusted with hydrochloric acid or sodium hydroxide, if required.
8) Volume was made up with water and further filled and packed in suitable container

### Example No. 3

| Sr. No. | Ingredients | Mg/ml | % w/w |
|---|---|---|---|
| 1 | Triamcinolone acetonide | 40 | 4 |
| 2 | Sodium chloride | 6.6 | 0.66 |
| 3 | Carboxymethylcellulose sodium | 6.3 | 0.63 |
| 4 | Polysorbate 80 | 0.4 | 0.04 |
| 5 | Benzalkonium chloride | 20.4 | 0.01 |
| 6 | Sodium Hydroxide | q.s | q.s |
| 7 | Hydrochloric acid | q.s | q.s |
| 8 | Water for injection | q.s to 1 ml | q.s |

### Manufacturing Process—

1) In S.S tank, under stirring sodium chloride and carboxymethylcellulose sodium were dissolved in water.
2) Benzalkonium chloride was added in step 1 solution. Then pH was adjusted with sodium hydroxide.
3) Polymer solution from step 2 was autoclaved using heat sterilization.
4) In a separate S.S. tank, Polysorbate 80 was dissolved in water and sterilized through filter, to this triamcinolone acetonide was added.
5) The API phase of step 4 is homogenized using homogenizer.
6) The homogenized solution of step 4 is aseptically mixed with polymer phase of step 3.
7) pH is adjusted with hydrochloric acid or sodium hydroxide, if required.
8) Volume was made up with water and further filled and packed in suitable container.

The injectable suspension stability is evaluated against various parameters and following table represent result for stability studies performed at different set of temperature and relative humidity.

### Batch No: TAS/R/40/040

TABLE NO 6

| | Product Name: | | | | | | |
|---|---|---|---|---|---|---|---|
| | Triamcinolone Acetonide Injectable Suspension 40 mg/mL | | | | | | |
| | Batch Size: | | | | | | |
| | 1000 mL | | | | | | |
| | Strategy | | | | | | |
| | Finalized strategy prototype batch | | | | | | |
| | Batch No. | | | | | | |
| | TAS/R/40/040 | | | | | | |
| | | 40° C./75% RH | | | | | |
| Condition | Initial | 1 Month | 2 Month | 3 Month | | 6 Month | |
| Orientation | | Upright | Upright | Upright | Inverted | Upright | Inverted |
| Appearance | White to off- | White to off- | White to off- | White to off- | | White to off- | |
| | white aqueous | white aqueous | white aqueous | white aqueous | | white aqueous | |
| | suspension | suspension | suspension | suspension | | suspension | |
| pH | 6.05 | 5.75 | 5.59 | 5.45 | 5.46 | 5.48 | 5.57 |
| Osmolality | 305 | 326 | 328 | 322 | 317 | 325 | 327 |
| (Miliosmol/kg) | | | | | | | |
| Viscosity (RPM | 25.6 | 16.2 | 12.4 | 10.3 | 12.1 | 7.23 | 7.95 |
| 20, Spindle S00) | | | | | | | |

Assay (By HPLC)

| | | | | | | | |
|---|---|---|---|---|---|---|---|
| Triamcinolone | 99.0 | 97.8 | 98.2 | 96.4 | 96.7 | 95.6 | 95.5 |
| Acetonide | | | | | | | |
| Benzyl alcohol | 98.7 | 98.7 | 99.2 | 97.2 | 96.8 | 101.6 | 99.8 |
| Limit of | ND | ND | 0.11 | 0.091 | 0.105 | 0.104 | 0.128 |
| Benzaldehyde | | | | | | | |

Related Substances (By HPLC)

| | | | | | | | |
|---|---|---|---|---|---|---|---|
| Triamcinolone | ND | ND | ND | ND | ND | ND | ND |
| 21-Hydrated | 0.03 | 0.04 | 0.039 | 0.045 | 0.041 | 0.089 | 0.072 |
| aldehyde | | | | | | | |
| Delta-14- | 0.03 | 0.03 | 0.033 | 0.028 | 0.027 | 0.026 | 0.024 |
| triamcinolone | | | | | | | |
| 1,2-dihydro | 0.01 | BLQ | BLQ | ND | ND | ND | ND |
| triamcinolone | | | | | | | |
| acetonide | | | | | | | |
| 9alpha-chloro | 0.01 | 0.02 | 0.017 | BLQ | BLQ | BLQ | BLQ |
| analog | | | | | | | |
| Triamcinolone | 0.012 | ND | ND | ND | ND | ND | ND |
| acetonide acetate | | | | | | | |
| Single maximum | 0.02 | 0.02 | 0.019 | 0.014 | 0.014 | 0.035 | 0.028 |
| individual | | | | | | | |
| unspecified | | | | | | | |
| degradation | | | | | | | |
| impurity | | | | | | | |
| Total impurities | 0.12 | 0.11 | 0.12 | 0.1 | 0.09 | 0.16 | 0.14 |

Dissolution using Type II Apparatus (Dissolution
parameters: water + SLS (0.35%), 250 mL 50 rpm, Paddle)

| Time points | % Release | | | | | | |
|---|---|---|---|---|---|---|---|
| 0 | 0 | NP | NP | NP | 0 | NP | 0 |
| 15 | 77 | | | | 67 | | 67 |
| 30 | 86 | | | | 78 | | 78 |
| 60 | 92 | | | | 86 | | 88 |
| 120 | 94 | | | | 89 | | 91 |
| 150 | NP | | | | NP | | 92 |
| Recovery | 98 | | | | 90 | | 91 |

ND: Not detected;
BLQ: Below limit of quantitation;
NP: Not performed

TABLE NO 7

| | Product Name: | | | | | | | | |
|---|---|---|---|---|---|---|---|---|---|
| | Triamcinolone Acetonide Injectable Suspension 40 mg/mL | | | | | | | | |
| | Batch Size: | | | | | | | | |
| | 1000 mL | | | | | | | | |
| | Strategy | | | | | | | | |
| | Finalized strategy prototype batch | | | | | | | | |
| | Batch No. | | | | | | | | |
| | TAS/R/40/040 | | | | | | | | |
| | | 30° C./65% RH | | | | 25° C./60% RH | | | |
| Condition | Initial | 3 Month | | 6 Month | | 3 Month | | 6 Month | |
| Orientation | | Upright | Inverted | Upright | Inverted | Upright | Inverted | Upright | Inverted |
| Appearance | White to off- | White to off- | | White to off- | | White to off- | | White to off- | |
| | white aqueous | white aqueous | | white aqueous | | white aqueous | | white aqueous | |
| | suspension | suspension | | suspension | | suspension | | suspension | |
| pH | 6.05 | 5.52 | 5.53 | 5.76 | 5.56 | 5.62 | 5.54 | 5.58 | 5.58 |
| Osmolality | 305 | 322 | 320 | 325 | 324 | 320 | 324 | 325 | 325 |
| (Miliosmol/kg) | | | | | | | | | |
| Viscosity (RPM | 25.6 | 18.4 | 20 | 16 | 15.9 | 21.9 | 21.2 | 19.3 | 21.8 |
| 20, Spindle S00) | | | | | | | | | |

Assay (By HPLC)

| | | | | | | | | | |
|---|---|---|---|---|---|---|---|---|---|
| Triamcinolone | 99.0 | 97.5 | 99.2 | 97.6 | 96.6 | 98 | 97.2 | 98.4 | 98.6 |
| Acetonide | | | | | | | | | |
| Benzyl alcohol | 98.7 | 98.6 | 98.2 | 102.5 | 102.1 | 97.7 | 97.6 | 102.6 | 103 |
| Limit of | ND | 0.06 | 0.06 | ND | ND | 0.05 | 0.05 | ND | ND |
| Benzaldehyde | | | | | | | | | |

Related Substances (By HPLC)

| | | | | | | | | | |
|---|---|---|---|---|---|---|---|---|---|
| Triamcinolone | ND | ND | ND | ND | ND | ND | ND | ND | ND |
| 21-Hydrated | 0.03 | 0.029 | 0.027 | 0.051 | 0.041 | 0.025 | 0.024 | 0.041 | 0.038 |
| aldehyde | | | | | | | | | |
| Delta-14- | 0.03 | 0.027 | ND | 0.025 | 0.026 | 0.03 | 0.03 | 0.027 | 0.03 |
| triamcinolone | | | | | | | | | |
| 1,2-dihydro | 0.01 | ND | BLQ | ND | ND | ND | ND | ND | ND |
| triamcinolone | | | | | | | | | |
| acetonide | | | | | | | | | |
| 9alpha-chloro | 0.01 | BLQ | ND | BLQ | BLQ | BLQ | BLQ | BLQ | BLQ |
| analog | | | | | | | | | |
| Triamcinolone | 0.012 | ND | ND | ND | ND | ND | ND | ND | ND |
| acetonide acetate | | | | | | | | | |
| Single maximum | 0.02 | 0.013 | 0.013 | 0.027 | 0.026 | 0.012 | 0.014 | 0.024 | 0.029 |
| individual | | | | | | | | | |
| unspecified | | | | | | | | | |
| degradation | | | | | | | | | |
| impurity | | | | | | | | | |
| Total impurities | 0.12 | 0.08 | 0.08 | 0.12 | 0.11 | 0.08 | 0.08 | 0.11 | 0.11 |

Dissolution using Type II Apparatus (Dissolution
parameters: water + SLS (0.35%), 250 mL 50 rpm, Paddle)

| Time points | | | | | | | | | |
|---|---|---|---|---|---|---|---|---|---|
| 0 | 0 | NP | NP | NP | NP | NP | 0 | NP | 0 |
| 15 | 77 | | | | | | 71 | | 72 |
| 30 | 86 | | | | | | 82 | | 84 |
| 60 | 92 | | | | | | 89 | | 90 |
| 120 | 94 | | | | | | 93 | | 92 |
| 150 | NP | | | | | | NP | | 92 |
| Recovery | 98 | | | | | | 93 | | 93 |

ND: Not detected;
BLQ: Below limit of quantitation;
NP: Not performed

### Conclusion from Table 6 and Table 7

From the above stability data, it was observed that there is no change in physical appearance of Triamcinolone Acetonide Injectable Suspension. No further decrease in pH was observed from 3 Months at all storage conditions. Viscosity at 6 Month was found to be around 7.0 to 8.0 cPs at accelerated storage condition. However about it remained in desirable range of 19 to 22 cPs at long term storage conditions. There is no significant change in Osmolality, Particle size distribution, Assay, Related substance data at 6 months accelerated and long term stability conditions at upright as well as inverted orientations. Dissolution profiles (by using USP Type II apparatus—Paddle) were found to be similar at initial and 6 months accelerated and long term stability storage condition.

Batch was manufactured at higher scale, reproducible to batch no. TAS/R/40/040 having initial parameters similar and it was found to be stable at accelerated and long term storage conditions a well. Results are tabulated in below table.

### Batch No: TAS/R/40/043

TABLE NO 8

| | Product Name: | | | | | | | | |
|---|---|---|---|---|---|---|---|---|---|
| | Triamcinolone Acetonide Injectable Suspension 40 mg/mL | | | | | | | | |
| | Strategy | | | | | | | | |
| | Reproducible batch of batch no: TAS/R/40/040 | | | | | | | | |
| | Batch No. | | | | | | | | |
| | TAS/R/40/043 | | | | | | | | |
| | Batch size | | | | | | | | |
| | 4.0 L | | | | | | | | |
| | | 40° C./75% RH | | | | | | | |
| Condition | Initial | 1 Month | | 2 Month | | 3 Month | | 6 Month | |
| Orientation | | Upright | Inverted | Upright | Inverted | Upright | Inverted | Upright | Inverted |
| Fill Volume | | 10 ml in 10 ml | | 10 ml in 10 ml | | 10 ml in 10 ml | | 10 ml in 10 ml | |
| Appearance | White to off- | White to off- | | White to off- | | White to off- | | White to off- | |
| | white aqueous | white aqueous | | white aqueous | | white aqueous | | white aqueous | |
| | suspension | suspension | | suspension | | suspension | | suspension | |
| pH | 6.0 | 6.0 | 6.0 | 5.8 | 5.9 | 5.8 | 5.8 | 5.9 | 5.93 |
| Osmolality | 334 | 328 | 328 | 321 | 320 | 322 | 326 | 325 | 322 |
| (Miliosmol/kg) | | | | | | | | | |
| Viscosity (RPM | 28.3 | 18.5 | 19 | 14.9 | 14.6 | 12.9 | 13.4 | 9.18 | 10.1 |
| 20, Spindle S00) | | | | | | | | | |

Assay (By HPLC)

| | | | | | | | | | |
|---|---|---|---|---|---|---|---|---|---|
| Triamcinolone | 98.8 | 97.6 | 97.6 | 100.3 | 99.5 | 98.6 | 100.1 | 100.4 | 99.2 |
| Acetonide | | | | | | | | | |
| Benzyl alcohol | 101.2 | 100.8 | 99.5 | 101.6 | 101.4 | 101.1 | 101.0 | 105.1 | 103.3 |
| Limit of | ND | ND | ND | 0.11 | 0.11 | 0.10 | 0.08 | ND | ND |
| Benzaldehyde | | | | | | | | | |

Related Substances (By HPLC)

| | | | | | | | | | |
|---|---|---|---|---|---|---|---|---|---|
| Triamcinolone | ND | ND | ND | ND | ND | ND | ND | ND | ND |
| 21-Hydrated | 0.042 | 0.044 | 0.043 | 0.047 | 0.049 | 0.034 | 0.031 | 0.058 | 0.056 |
| aldehyde | | | | | | | | | |
| Delta-14- | 0.037 | 0.03 | 0.029 | 0.03 | 0.03 | 0.017 | 0.016 | 0.031 | 0.029 |
| triamcinolone | | | | | | | | | |
| 1,2-dihydro | BLQ | BLQ | BLQ | 0.011 | 0.011 | ND | ND | ND | ND |
| triamcinolone | | | | | | | | | |
| acetonide | | | | | | | | | |
| 9alpha-chloro | 0.015 | 0.014 | 0.011 | 0.013 | 0.011 | BLQ | ND | BLQ | BLQ |
| analog | | | | | | | | | |
| Triamcinolone | ND | ND | ND | ND | ND | ND | ND | ND | ND |
| acetonide acetate | | | | | | | | | |
| Single maximum | 0.021 | 0.019 | 0.016 | 0.017 | 0.018 | 0.013 | 0.009 | 0.024 | 0.026 |
| individual | | | | | | | | | |
| unspecified | | | | | | | | | |
| degradation | | | | | | | | | |
| impurity | | | | | | | | | |
| Total impurities | 0.13 | 0.12 | 0.11 | 0.13 | 0.13 | 0.08 | 0.07 | 0.13 | 0.13 |

Dissolution using Type II Apparatus (Dissolution
parameters: water + SLS (0.35%), 250 mL 50 rpm, Paddle)

| Time points | % Release | | | | | | | | |
|---|---|---|---|---|---|---|---|---|---|
| 0 | 0 | NP | 0 | NP | 0 | NP | 0 | NP | 0 |
| 15 | 76 | | 79 | | 79 | | 74 | | 77 |
| 30 | 87 | | 88 | | 89 | | 86 | | 86 |
| 60 | 91 | | 93 | | 94 | | 91 | | 91 |
| 120 | 93 | | 95 | | 97 | | 93 | | 93 |
| 150 | NP | | NP | | NP | | NP | | 94 |
| Recovery | 92 | | 95 | | 96 | | 93 | | 94 |

ND: Not detected;
BLQ: Below limit of quantitation;
NP: Not performed

TABLE 9

| | Product Name: | | | | |
|---|---|---|---|---|---|
| | Triamcinolone Acetonide Injectable Suspension 40 mg/mL | | | | |
| | Strategy | | | | |
| | Reproducible batch of batch no: TAS/R/40/040 | | | | |
| | Batch No. | | | | |
| | TAS/R/40/043 | | | | |
| | Batch size | | | | |
| | 4.0 L | | | | |
| | | 25° C./60% RH | | | |
| Condition | Initial | 3 Month | | 6 Month | |
| Orientation | | Upright | Inverted | Upright | Inverted |
| Fill Volume | | 10 ml in 10 ml | | 10 ml in 10 ml | |
| Appearance | White to off- | White to off- | | White to off- | |
| | white aqueous | white aqueous | | white aqueous | |
| | suspension | suspension | | suspension | |
| pH | 6.0 | 5.88 | 5.85 | 6.08 | 6.08 |
| Osmolality | 334 | 325 | 325 | 319 | 323 |
| (Miliosmol/kg) | | | | | |
| Viscosity (RPM | 28.3 | 22 | 21.1 | 19.3 | 20.7 |
| 20, Spindle S00) | | | | | |

Assay (By HPLC)

| | | | | | |
|---|---|---|---|---|---|
| Triamcinolone | 98.8 | 100.0 | 99.1 | 98.5 | 101.0 |
| Acetonide | | | | | |
| Benzyl alcohol | 101.2 | 101.5 | 101.3 | 104.1 | 104.8 |
| Limit of Benzaldehyde | ND | 0.08 | 0.07 | 0.12 | ND |

Related Substances (By HPLC)

| | | | | | |
|---|---|---|---|---|---|
| Triamcinolone | ND | ND | ND | ND | ND |
| 21-Hydrated aldehyde | 0.042 | 0.028 | 0.027 | 0.033 | 0.042 |
| Delta-14- | 0.037 | 0.018 | 0.017 | 0.024 | 0.032 |
| triamcinolone | | | | | |
| 1,2-dihydro | BLQ | BLQ | BLQ | ND | ND |
| triamcinolone | | | | | |
| acetonide | | | | | |
| 9alpha-chloro analog | 0.015 | BLQ | BLQ | BLQ | 0.011 |
| Triamcinolone | ND | ND | ND | ND | ND |
| acetonide acetate | | | | | |
| Single maximum | 0.021 | 0.011 | 0.011 | 0.023 | 0.028 |
| individual unspecified | | | | | |
| degradation impurity | | | | | |
| Total impurities | 0.13 | 0.07 | 0.07 | 0.09 | 0.13 |

Dissolution using Type II Apparatus (Dissolution
parameters: water + SLS (0.35%), 250 mL 50 rpm, Paddle)

| Time points | % Release | | | | |
|---|---|---|---|---|---|
| 0 | 0 | NP | 0 | NP | 0 |
| 15 | 76 | | 79 | | 76 |
| 30 | 87 | | 87 | | 84 |
| 60 | 91 | | 90 | | 90 |
| 120 | 93 | | 93 | | 94 |
| 150 | NP | | NP | | 95 |
| Recovery | 92 | | 92 | | 95 |

ND: Not detected;
BLQ: Below limit of quantitation;
NP: Not performed

Conclusion from Table 8 and 9: From the above stability data, it was observed that there is no change in physical appearance of Triamcinolone Acetonide Injectable suspension. Viscosity at 6 Month time point dropped in a range of 9.0 to 10.0 cPs at 6 Month accelerated storage condition which remained in a range of 19 to 20 cPs at long term storage condition. There is no significant change in pH, Osmolality, Particle size distribution, Assay, Related substance data at 6 months accelerated and long term stability conditions at upright as well as inverted orientations. Dissolution profiles (by using USP Type II apparatus—Paddle) were also found to be similar at initial and 6 months accelerated and long term stability storage condition. Hence increasing the batch size showed comparable physicochemical parameter.

We claim:

1) A stable injectable composition comprising triamcinolone acetonide and other pharmaceutical excipients.

2) The stable injectable composition of as claimed in claim 1, wherein triamcinolone acetonide is used in the range of 1% to 8%.

3) The stable injectable composition of as claimed in claim 1, wherein said composition is stable over time at controlled room temperature between 20° C. to 25° C. and compatible to the glass packaging material.

4) The stable injectable composition of as claimed in claim 1, wherein said composition has viscosity in the range of 10 to 30 cps.

5) The stable injectable composition of as claimed in claim 1, wherein said composition has osmolality in the range of 270 to 370 mOsm/kg.

6) The stable injectable composition of as claimed in claim 1, wherein said composition is preservative free.

7) The stable injectable composition of as claimed in claim 1, wherein the average particle size of triamcinolone acetonide is 5 μm to 20 μm.

8) The stable injectable composition as claimed in claim 1, wherein pharmaceutical acceptable excipients are selected from the group consisting of tonicity adjusting agent, viscosity modifying agent, wetting agent, preservative, pH adjusting agent and combination thereof.

9) A process for the preparation of a stable injectable composition as claimed in claim 1, comprising of:
a) preparing polymer phase comprising of sodium chloride, carboxymethylcellulose sodium and water;
b) adding preservative to polymer phase of step a) and adjusting pH with sodium hydroxide or hydrochloric acid, followed by moist heat sterilization at 121° C. and at 15 psi pressure for 15 minutes;
c) preparing a solution comprising of polysorbate 80 and water, followed by sterilization by filtration;
d) preparing API phase by mixing solution of step (c) and sterile triamcinolone acetonide followed by homogenization to reduce particle size in the range of 7 μm to 15 μm;
e) mixing both polymer phase of step (b) and API phase of step (d), followed by final pH adjustment and volume make up to prepare a homogenize suspension of triamcinolone acetonide.
